(19) United States
(12) Patent Application Publication
(10) Pub. No.: US 2008/0261897 A1
(43) Pub. Date: Oct. 23, 2008

(54) Formulation Containing a Polyphenol-Containing Composition and Isomaltulose

(76) Inventors: Tillmann Dorr, Hohen-Sulzen (DE); Stephan Hausmanns, Wiesbaden (DE); Jorg Kowalczyk, Eisenberg/Steinborn (DE); Martina Becker, Lorrach (DE); Ulrich Killeit, Grenzach-Wyhlen (DE)

Correspondence Address: OSTROLENK FABER GERB & SOFFEN, 1180 AVENUE OF THE AMERICAS, NEW YORK, NY, 100368403, US

(21) Appl. No.: 12/094,253

(22) PCT Filed: Nov. 23, 2006
(86) PCT No.: PCT/EP2006/011219

§ 371 Date: May 19, 2008

(30) Foreign Application Priority Data
Nov. 25, 2005 (DE) 10 2005 056 652.9

(51) Int. Cl. A61K 31/7016; A23L 2/52

(52) U.S. Cl. 514/25; 426/590

(57) ABSTRACT

The present invention relates to formulations containing isomaltulose and a polyphenol-containing composition, uses of same and the use of isomaltulose for masking unwanted taste components, in particular bitter substances, in formulations, in particular in formulations containing tea extracts.

The present invention relates to formulations containing polyphenol-containing compositions and isomaltulose, uses of the above-mentioned formulation and also the use of isomaltulose as taste-masking agent in a formulation containing a polyphenol-containing composition.

In a great multiplicity of foods and articles consumed for pleasure and also, in particular, in drinks, there are a number of bitter substances, some wanted, but some unwanted, that is to say compounds which give rise to a bitter taste for the consumers, in particular human consumers. Typical bitter substances are, for example, substances from the class of glycosides, but also isoprenoids and catechins which are the base substance of a multiplicity of oligomeric or polymeric tannins. Polyphenol-based bitter substances are also present in teas or tea extracts. Such extracts also include extracts of green tea, for example the extract, also named TEAVIGO® from leaves of green tea which contains at least 90% epigallocatechin gallate (EGCG). Although this already has a relatively acceptable degree of bitterness, for some consumers it is still too bitter. Tannins such as tannic acids generally have a bitter taste which can be a nuisance in drinks and goods, articles consumed for pleasure or medicaments. U.S. Pat. No. 5,902,628 describes shortening a persistent sweet aftertaste of the intense sweetener sucralose by admixing tannic acids. Although the tannic acid according to this publication beneficially affects the strongly sweet aftertaste of sucralose which is considered a disadvantage, nevertheless the bitter taste of tannic acid in many applications proves to be disadvantageous, despite the simultaneous presence of sucralose.

The reducing disaccharide ketose isomaltulose (6-O-α-D-glucopyranosylfructose) which occurs naturally, for example in honey, and is also known to those skilled in the art as Palatinose®, is principally used as starting material for producing isomalt, a virtually equimolar mixture of the diastereomers 6-O-α-D-glucopyranosyl-D-sorbitol (1,6-GPS) and 1-O-α-D-glucopyranosyl-D-mannitol (1,1-GPM). As sweetening agent, isomaltulose, owing to its low sweetening power and the taste resulting therefrom is principally used in combination with sugar replacers and/or sweeteners in foods. Owing to delayed breakdown of isomaltulose which takes place first in the region of the small intestine, isomaltulose is also used in special foods for athletes in order to maintain oxidative metabolism.

Isomaltulose crystallizes in the form of a monohydrate. The solubility of isomaltulose in water is 0.49 g of anhydrous isomaltulose per g of water. Isomaltulose has advantageous acariogenic properties, since it is scarcely broken down by the human oral flora. Isomaltulose is only cleaved with a delay by the glucosidases of the human small intestinal wall, wherein the resultant breakdown products glucose and fructose are resorbed. This results in a slow rise in blood glucose, compared with rapidly digestible carbohydrates. Isomaltulose, in contrast to rapidly digestible, high-glycemic foods, scarcely requires insulin for metabolism.

Isomaltulose is also used in part to mask the unpleasant taste of other foods. EP 0 809 939 A1 describes a yoghurt which contains lactic acid bacteria and bifido bacteria which also contains refined fish oil having a high fraction of unsaturated fatty acids, and also a sweetening agent, for example isomaltulose. The addition of isomaltulose is intended to prevent the development of the typical fish taste and fish odor. JP 63152950 A2 describes the production of vegetable jelly products, using vegetables and a gelling agent, wherein isomaltulose and other additives such as cinnamon are used in order to mask the unpleasant odor of vegetable components.

DE 690 005 48 T2 describes sweetener compositions containing sucralose and isomaltulose, wherein these compositions have a synergistic effect, that is to say exhibit a greater sweetening power than would be expected by simple addition of the sweetening power exhibited by the sweetener components. The sweetener mixture can be used, for example, for producing drinks and confectionery products.

However, there is still a requirement for a teaching according to which the unwanted bitter taste of bitter substances, in particular polyphenol-based bitter substances, in formulations, that is say, for example, foods, drinks, medicaments or articles consumed for pleasure, can be masked and thus as far as possible removed from perception by the consumer.

Taste here is taken to mean the chemical sense of humans for perception and differentiation of foodstuffs. Humans can essentially differentiate four taste qualities: sweet, sour, bitter and salty. The taste stimulus occurs by activation of a taste sensory cell by the addition of molecules of a taste substance to receptor molecules. A taste stimulus can be expressed differently not only in its quality, for example whether it is sweet, sour, bitter, salty, or a mixture thereof, in its intensity, that is to say the taste strength, and also in its duration.

The technical problem therefore underlying the present invention is to provide a teaching according to which the unwanted bitter taste of bitter substances, in particular polyphenol-containing compositions, is masked in a formulation, that is say is no longer perceived, or is perceived only to a greatly reduced extent, by the consumer, in particular human or animal consumer, in particular a bitter taste impression during and after consumption is no longer perceived.

The present invention solves its underlying technical problem by providing the teaching of using isomaltulose in order to mask the bitter taste of polyphenol-containing mixtures, in particular compositions. The invention solves its underlying problem, in particular, also, by providing a formulation containing a polyphenol-containing composition and isomaltulose. The invention therefore relates to the teaching of using isomaltulose together with an, in particular bitter tasting, polyphenol-containing composition, wherein the isomaltulose masks the otherwise bitter taste of the polyphenol-containing composition. According to the invention the isomaltulose therefore acts as taste-masking agent, which acts not only during the consumption of the polyphenol-containing composition, or of a formulation which contains a polyphenol-containing composition, but also masks the bitter taste which is otherwise also perceived by the consumer after consumption, that is say the aftertaste. Advantageously, by this means a formulation can be provided which, owing to the isomaltulose used, has sweetening power, but is nonetheless protective to teeth, acariogenic, non-laxative, and suitable for diabetics.

In the context of the present invention, taste masking of a polyphenol-containing composition is taken to mean that a taste of the polyphenol-containing composition which is perceived as bitter by test persons of competence in the art in a comparison formulation with polyphenol-containing composition and comparison sweetener is perceived in a statistically meaningful manner to a significantly reduced extent, preferably no longer at all, when this polyphenol-containing composition is present in a formulation of the invention together with isomaltulose instead of with the comparison sweetener.

In the context of the present invention, a bitter tasting polyphenol-containing composition is taken to mean a composition which contains polyphenols, in particular polyphenol derivatives, in an amount which is considered as bitter tasting by test persons of competence in the art in a statistically meaningful manner. In a preferred embodiment, a polyphenol-containing composition of the present invention is a composition which has catechin derivatives, preferably at least 90% by weight, in particular at least 94% by weight, in each case based on the dry matter of the composition. In a particularly preferred embodiment, the polyphenol-containing composition of the present invention is a composition which contains at least 90% by weight epigallocatechin gallate, hereinafter also termed EGCG (polyphenol(-)-epigallocatechin-3-gallate), preferably at least 94% by weight EGCG (in each case based on dry matter of the polyphenol-containing composition).

In a particularly preferred embodiment of the present invention, this relates to a formulation which contains a polyphenol-containing composition and isomaltulose, wherein the polyphenol-containing composition has a fraction of at least 90% by weight, preferably at least 94% by weight, of EGCG, in each case based on the weight of the dry matter of the polyphenol-containing composition.

In a further preferred embodiment, the invention relates to an above-mentioned formulation, wherein the polyphenol-containing composition has a fraction of at most 2.5% by weight of caffeine, preferably at most 0.1% by weight of caffeine, in each case based on the weight of the dry matter of the polyphenol-containing composition.

In a further preferred embodiment, the present invention relates to an above-mentioned formulation, wherein the polyphenol-containing composition has a fraction of at most 2.5% by weight of gallocatechin gallate (GCG), based on the weight of the dry matter of the polyphenol-containing composition.

The invention relates in a further preferred embodiment to a formulation of the above-mentioned type, wherein the polyphenol-containing composition has a fraction of at most 5.0% by weight of epicatechin gallate (ECG), preferably at most 3.0% by weight ECG, in each case based on the weight of the dry matter of the polyphenol-containing composition.

The invention relates in a further preferred embodiment to an above-mentioned formulation wherein the polyphenol-containing composition has a fraction of at most 0.1% by weight of gallic acid, based on the weight of the dry matter of the polyphenol-containing composition.

The invention further relates in a further preferred embodiment to a formulation of the above-mentioned type, wherein the polyphenol-containing composition, apart from EGCG, has a fraction of at most 5.0% by weight of other polyphenols and catechins such as gallocatechin gallate (GCG), catechin gallate (CG), epicatechin gallate (ECG), epigallocatechin (EGC), gallocatechin (GC) and epicatechin (EC), in each case based on the weight of the dry matter of the polyphenol-containing composition. In a preferred embodiment, the polyphenol-containing composition is TEAVIGO®.

TEAVIGO® is a highly purified extract of leaves of the green tea Camellia sinensis and has at least 90% by weight, preferably at least 94% by weight, EGCG and a maximum of 0.1% by weight caffeine.

The invention relates, furthermore, in a further preferred embodiment, of course also to the use of isomaltulose in a formulation containing a polyphenol-containing composition for taste masking. In a particularly preferred embodiment, the taste masking is taste masking of a polyphenol-containing composition.

In a further preferred embodiment of the present invention, the invention relates to the use of a formulation of the present invention for increasing fat burning in mammals, in particular in humans. In a further preferred embodiment, the present invention relates to the use of an above-mentioned formulation for supporting fat metabolism in mammals, in particular in humans.

In a further preferred embodiment, the present invention relates to the use of an above-mentioned formulation for reducing the fat mass in mammals, in particular in humans.

In a further preferred embodiment of the present invention, the present invention relates to the use of an above-mentioned formulation for producing a medicament for increasing fat burning and/or for supporting fat metabolism and/or for reducing the fat mass in mammals, in particular in humans.

The invention also relates, furthermore, to an instant powder containing 50 to 80% by weight isomaltulose, 0.1 to 10% by weight EGCG, 0.1 to 10% by weight caffeine, and also 0.005 to 1.0% by weight B vitamins.

In the context of the present invention, mammals are taken to mean, in particular, humans, cats, dogs and horses. In the context of the present invention, B vitamins are taken to mean in particular vitamin B₁, vitamin B₂ and nicotinamide.

In a preferred embodiment, EGCG is applied in the formulations and uses according to the invention in such a manner that the effective dose of EGCG is from 0.14 to 25 mg/kg of bodyweight/day, preferably from 2.0 to 9.0 mg/kg of bodyweight/day, particularly preferably from 4.0 to 9.0 mg/kg of bodyweight/day, and in particular from 4.0 to 4.5 mg/kg of bodyweight/day,

In a preferred embodiment of the present invention, the polyphenol-containing composition is a plant extract, in particular an isolated and purified, in particular highly purified, plant extract, in particular a tea extract.

In a further preferred embodiment, the extract is an extract of black tea, of Oolong tea, or of green tea, preferably an extract of leaves of green tea, that is to say Camellia sinensis.

In a further preferred embodiment, the extract of the above-mentioned type contains additionally natural or nature-identical odor and/or taste substances.

According to the invention it is preferably provided that isomaltulose is used in order to mask the bitter taste of the polyphenol-containing composition present in a formulation of the present invention, that is to say to make it unrecognizable or not perceivable, or perceivable to a greatly reduced extent. In a preferred embodiment of the present invention, such a formulation is a composition which is intended or suitable for human or animal consumption, for example a drink powder, an instant powder, a product for oral hygiene, a cosmetics product, a dry flavor formula, a foodstuff, food or article consumed for pleasure, or a feed. In a preferred embodiment, this formulation can be present in dry form, preferably in free-flowing form, in particular in agglomerated, pulverized and/or freeze-dried form.

The invention also relates, in particular, to possibly coated formulations which are in the form of compactates, capsules or tablets as foods, articles consumed for pleasure, foodstuffs or medicaments, an instant powder, as drink powder, as drinks or as cosmetics and also products for oral hygiene.

Of course, the formulation can also be used in order to produce drinks, that is to say to act as instant powder, in particular instant tea powder or as dry flavor formula. The effects observed according to the invention are then found in the drink produced by means of the formulation according to the invention, for example a tea drink, which is likewise subject matter of the present teaching.

In a further preferred embodiment, in the formulation according to the invention food-compatible acids and/or salts can also be present. In a further preferred embodiment of the invention, intense sweeteners, for example saccharin, saccharin-Na, saccharin-K, saccharin-Ca, sodium cyclamate, calcium cyclamate, acesulfame-K, aspartame, dulcin, stevioside, neohesperedine dihydrochalcone or sucralose can also be present in the formulation according to the invention.

In a further preferred embodiment, antioxidants, stabilizers, minerals and/or trace elements can be present in the formulation. In a further preferred embodiment, vitamins can also be present in the formulation according to the invention, for example natural or synthetic vitamins, in particular vitamin A, vitamin B₁, vitamin B₂, vitamin B₃, vitamin B₅, vitamin B₆, vitamin B12, vitamin B complex, nicotinamide, vitamin C, vitamin E, vitamin F and vitamin K.

In a further preferred embodiment, the formulations according to the invention can contain flow enhancers, for example silica.

In a further preferred embodiment, the formulations according to the invention can contain natural and/or synthetic dyes, for example dyes of plant origin, animal origin, inorganic pigments, products of enzymatic browning, products of non-enzymatic browning and carbohydrate heating products. As synthetic dyes, use can be made of, for example, azo compounds, triphenylmethane compounds, indigoid compounds, xanthene compounds or quinoline compounds.

In a further preferred embodiment, the present invention relates to an above-mentioned formulation, wherein the fraction of polyphenol-containing composition in the formulation is 0.1 to 10% by weight, preferably 0.3 to 10% by weight, in particular 0.3 to 5% by weight, preferably 0.6 to 9% by weight, in particular 0.7 to 8% by weight, more preferably 0.9 to 7% by weight, in particular 1 to 5% by weight (in each case based on the dry matter of the formulation).

In a further preferred embodiment, the present invention relates to an above-mentioned formulation, wherein the fraction of isomaltulose in the formulation is preferably 30 to 85% by weight, preferably 30 to 80% by weight, preferably 50 to 80% by weight, in particular 35 to 75% by weight, in particular 40 to 70% by weight, preferably 45 to 65% by weight, in particular 50 to 61% by weight (in each case based on the dry matter of the formulation).

In a further preferred embodiment, the present invention relates to an above-mentioned formulation, wherein the fraction of food-compatible acids in the formulation is 5 to 10% by weight (based on the dry matter of the formulation).

In a further preferred embodiment, the present invention relates to an above-mentioned formulation, wherein the fraction of intense sweeteners in the formulation is 0.1 to 1% by weight (based on the dry matter of the formulation).

In a further preferred embodiment, the present invention relates to an above-mentioned formulation, wherein the fraction of taste substances in the formulation is 0.5 to 30% by weight (based on the dry matter of the formulation).

In a further preferred embodiment, the formulation according to the invention, apart from isomaltulose, does not contain any further sugars, in particular does not contain sucrose, fructose and/or glucose. Preferably, isomaltulose is the sole sugar present in the formulation, in particular the sole sweetening agent present in the formulation.

In the context of the present invention, the term sweetening agent is taken to mean a substance which has sweetening power and is added, for example, to foods or drinks in order to give rise to a sweet taste. In the context of the present invention, the sweetening agents are sub-divided into sugars, such as isomaltulose, sucrose, glucose or fructose, which give body and sweetening power, and also “sweeteners”, that is to say substances which are not sugars, but nevertheless have sweetening power, which in turn are subdivided into “sugar replacers”, that is to say sweetening agents which have body and physiological calorific value in addition to sweetening power (bulking sweeteners) and “intense sweeteners”, that is to say substances which generally have a very high sweetening power, but do not have body and generally have no, or only very low, physiological calorific value.

In a particularly preferred embodiment, the formulation according to the invention is protective to teeth, acariogenic and/or dietetic.

According to the invention, however, in a further embodiment it can be provided that the isomaltulose is used together with intense sweeteners and/or if appropriate also sugar replacers, for example isomalt, in the formulation.

In a further preferred embodiment, the invention relates to the polyphenol-containing formulations according to the invention containing isomaltulose together with at least one, preferably strongly sweetening, carbohydrate, which preferably has an equivalent sweetening power of >0.5, based on a 10% strength sucrose solution, e.g. sucrose, fructose, glucose, invert sugar and/or oligofructose. The invention also relates to polyphenol-containing formulations according to the invention containing isomaltulose and glucose-containing, fructose-containing and/or leucrose-containing syrups, for example sucromalt. The invention also relates to polyphenol-containing formulations according to the invention containing isomaltulose and mixtures of sucrose, glucose and/or fructose with other carbohydrates.

The strongly masking sensory properties of isomaltulose, in particular in combination with strongly sweetening carbohydrates, deliver in polyphenol-containing, in particular in Teavigo-containing drink formulas, a balanced and rounded sweetness profile with a full mouthfeel, compared with products which are sweetened classically.

In particular, the preferred polyphenol-containing formulations according to the invention having a carbohydrate fraction, in particular isomaltulose fraction, of 30 to 85%, preferably 45 to 65%, in drinks exhibit a pronounced beneficial total sensory impression.

Further advantageous embodiments of the present invention are given by the subclaims.

The invention will be described in more detail with reference to an exemplary embodiment.

### EXAMPLE

Formula 1 (Comparative formula)

| | Instant powder | |
|---|---|---|
| Components | (g/kg) | rtd (g/200 ml) |
| Maltodextrin | 569.300 | 8.540 |
| Citric acid, anhydrous | 66.670 | 1.000 |
| powder | | |
| Trisodium citrate, | 12.500 | 0.188 |
| dihydrate powder | | |
| TWINSWEET ® | 3.330 | 0.050 |
| Grapefruit taste | 8.330 | 0.125 |
| TEAVIGO ® | 2.540 | 0.038 |
| Caffeine | 3.330 | 0.050 |
| CustoMix BE-P | 0.670 | 0.010 |
| Grapefruit 250L powder | 333.330 | 5.000 |
| Total | 1000.000 | 15.000 |

Formula 2 with sucrose (Comparative formula)

| | Instant powder | |
|---|---|---|
| Components | (g/kg) | rtd (g/200 ml) |
| Sucrose | 607.800 | 6.078 |
| Citric acid, anhydrous | 80.000 | 0.800 |
| powder | | |
| Trisodium citrate, | 14.400 | 0.144 |
| dihydrate powder | | |
| TWINSWEET ® | 3.000 | 0.030 |
| Grapefruit taste | 11.000 | 0.110 |
| TEAVIGO ® | 3.800 | 0.038 |
| Caffeine | 4.000 | 0.040 |
| CustoMix BE-P | 1.000 | 0.010 |
| Grapefruit 250L powder | 275.000 | 2.750 |
| Total | 1000.000 | 10.000 |

Formula 3 with isomaltulose

| | Instant powder | |
|---|---|---|
| Components | (g/kg) | rtd (g/200 ml) |
| Isomaltulose | 607.800 | 6.078 |
| Citric acid, anhydrous | 80.000 | 0.800 |
| powder | | |
| Trisodium citrate, | 14.400 | 0.144 |
| dihydrate powder | | |
| TWINSWEET ® | 3.000 | 0.030 |
| Grapefruit taste | 11.000 | 0.110 |
| TEAVIGO ® | 3.800 | 0.038 |
| Caffeine | 4.000 | 0.040 |
| CustoMix BE-P | 1.000 | 0.010 |
| Grapefruit 250L powder | 275.000 | 2.750 |
| Total | 1000.000 | 10.000 |

Formula 4 with isomaltulose

| | Instant powder | |
|---|---|---|
| Components | (g/kg) | rtd (g/250 ml) |
| Isomaltulose | 583.300 | 7.000 |
| Citric acid, anhydrous | 83.300 | 1.000 |
| powder | | |
| Trisodium citrate, | 15.000 | 0.180 |
| dihydrate powder | | |
| TWINSWEET ® | 4.200 | 0.050 |
| Grapefruit taste | 11.500 | 0.138 |
| TEAVIGO ® | 3.200 | 0.038 |
| Caffeine | 4.200 | 0.050 |
| CustoMix BE-P | 0.800 | 0.010 |
| Grapefruit 250L powder | 294.500 | 3.534 |
| Total | 1000.000 | 12.000 |

Formula 5 with isomaltulose and sucrose

| | Instant powder | |
|---|---|---|
| Components | (g/kg) | rtd (g/200 ml) |
| Isomaltulose | 407.800 | 4.078 |
| Sucrose | 200.000 | 2.000 |
| Citric acid, anhydrous | 80.000 | 0.800 |
| powder | | |
| Trisodium citrate, | 14.400 | 0.144 |
| dihydrate powder | | |
| TWINSWEET ® | 3.000 | 0.030 |
| Grapefruit taste | 11.000 | 0.110 |
| TEAVIGO ® | 3.800 | 0.038 |
| Caffeine | 4.000 | 0.040 |
| CustoMix BE-P | 1.000 | 0.010 |
| Grapefruit 250L powder | 275.000 | 2.750 |
| Total | 1000.000 | 10.000 |

Formula 6 with isomaltulose and fructose

| | Instant powder | |
|---|---|---|
| Components | (g/kg) | rtd (g/200 ml) |
| Isomaltulose | 457.800 | 4.578 |
| Fructose | 150.000 | 1.500 |
| Citric acid, anhydrous | 80.000 | 0.800 |
| powder | | |
| Trisodium citrate, | 14.400 | 0.144 |
| dihydrate powder | | |
| TWINSWEET ® | 3.000 | 0.030 |
| Grapefruit taste | 11.000 | 0.110 |
| TEAVIGO ® | 3.800 | 0.038 |
| Caffeine | 4.000 | 0.040 |
| CustoMix BE-P | 1.000 | 0.010 |
| Grapefruit 250L powder | 275.000 | 2.750 |
| Total | 1000.000 | 10.000 |

Formula 7 with isomaltulose and dry glucose

| | Instant powder | |
|---|---|---|
| Components | (g/kg) | rtd (g/200 ml) |
| Isomaltulose | 207.800 | 2.078 |
| Dry glucose (from Agrana) | 400.000 | 4.000 |
| Citric acid, anhydrous | 80.000 | 0.800 |
| powder | | |
| Trisodium citrate, | 14.400 | 0.144 |
| dihydrate powder | | |
| TWINSWEET ® | 3.000 | 0.030 |
| Grapefruit taste | 11.000 | 0.110 |
| TEAVIGO ® | 3.800 | 0.038 |
| Caffeine | 4.000 | 0.040 |
| CustoMix BE-P | 1.000 | 0.010 |
| Grapefruit 250L powder | 275.000 | 2.750 |
| Total | 1000.000 | 10.000 |

Formula 8 with isomaltulose/invert sugar syrup compound*

| | Instant powder | |
|---|---|---|
| Components | (g/kg) | rtd (g/200 ml) |
| Isomaltulose fraction in | 487.800 | 4.878 |
| the compound | | |
| Invert sugar fraction in | 120.000 | 1.200 |
| the compound | | |
| Citric acid, anhydrous | 80.000 | 0.800 |
| powder | | |
| Trisodium citrate, | 14.400 | 0.144 |
| dihydrate powder | | |
| TWINSWEET ® | 3.000 | 0.030 |
| Grapefruit taste | 11.000 | 0.110 |
| TEAVIGO ® | 3.800 | 0.038 |
| Caffeine | 4.000 | 0.040 |
| CustoMix BE-P | 1.000 | 0.010 |
| Grapefruit 250L powder | 275.000 | 2.750 |
| Total | 1000.000 | 10.000 |

*The compound was produced by agglomeration, wherein finely ground isomaltulose was used and the slightly diluted invert sugar solution (roughly the same parts of sucrose, glucose and fructose) having a dry matter content of approximately 60% was sprayed on as binder component.

For the above-mentioned formulas, the column “instant powder” gives the concentration of the formula component specified in each case in g/kg in the overall formulation. The column “rtd” (ready to drink) gives the specified concentration of the formula component in grams per 200 ml in a drink produced using the formulation. For Comparative Formula 1, per 200 ml of drink, 15 g of the instant powder were used, whereas in formula 2, 3, 5 to 8, use was made of 10 g of the instant powder according to the invention per 200 ml of drink, and in Formula 4, 12 g per 250 ml of drink.

TEAVIGO® is a highly purified extract of leaves of the green tea Camellia sinensis and has at least 90% by weight, preferably at least 94% by weight, EGCG and a maximum of 0.1% by weight of caffeine.

For production of the drink, the formulation was dissolved in water with stirring so as to be ready to drink.

The drink was presented to a trained panel of 15 testers for tasting.

The testers, in the context of a profile analysis with scale, had to evaluate the bitterness (on drinking and aftertaste), the sweetness impression, the mouthfeel, the flavor impression and the overall impression of the drinks produced from the formulas.

The attributes were defined as follows on a scale from 0 to 5:
Bitterness (on drinking): 0=unpleasantly bitter; 5=bitter note scarcely perceptible
Bitterness (aftertaste): 0=unpleasantly bitter; 5=bitter note scarcely perceptible
Sweetness impression: 0=unbalanced; 5=balanced, round
Mouthfeel: 0=unpleasant, 5=full bodied, pleasant
Aroma impression: 0=short, flat; 5=long-lasting, balanced
Overall impression: 0=unbalanced, qualitatively bad; 5=balanced, round, qualitatively good

The testers, in a statistically meaningful manner, established for the comparative formula, not only during the tasting, but also immediately thereafter, a significantly more bitter taste than for the formulas according to the invention. Further advantages were also demonstrated in the other attributes tested. In particular in the case of sweetness and overall impression, the products according to the invention containing the isomaltulose-carbohydrate mixtures exhibit very good sensory property profiles. The results of the profile analysis are summarized numerically in the table.

These results are surprising, since the formulas vary with respect to sweetening power. Sweetening power is apparently not the parameter responsible for hiding the bitter note, that is to say concealing the same amount of bitter substances, in particular of polyphenol-containing extract, that is TEAVIGO®. The data show that isomaltulose, together with a polyphenol-containing composition, effects a particularly beneficial sensory profile. If isomaltulose is used in combination with at least one strongly sweetening carbohydrate, for example fructose, glucose, sucrose and/or invert sugar, this gives a further improved, beneficial, sensory profile.

TABLE
Results of sensory profile analysis

| | | | | | Formula 5 | | Formula 7 | |
|---|---|---|---|---|---|---|---|---|
| | Formula 1 | Formula 2 | Formula 3 | Formula 4 | Isom./ | Formula 6 | Isom./Dry | Formula 8 |
| | Maltodextrin | Sucrose, | Isomaltulose | Isomaltulose | Sucr | Isom./Fruc. | glu. | Isom./Inverts. |
| Bitterness | 1.6 | 1.9 | 3.1 | 3.2 | 3.2 | 3.1 | 3.0 | 3.5 |
| on drinking | | | | | | | | |
| Bitterness | 2.0 | 2.3 | 3.9 | 3.8 | 4.2 | 4.3 | 4.3 | 4.4 |
| (aftertaste) | | | | | | | | |
| Sweetness | 2.4 | 3.1 | 2.9 | 2.7 | 3.5 | 3.8 | 3.3 | 3.9 |
| impression | | | | | | | | |
| Mouthfeel | 1.5 | 2.7 | 2.9 | 3.1 | 3.2 | 3.4 | 3.2 | 3.5 |
| Flavor | 3.1 | 3.2 | 3.7 | 3.6 | 3.8 | 4.0 | 3.7 | 4.1 |
| impression | | | | | | | | |
| Overall | 1.9 | 2.6 | 3.3 | 3.2 | 3.6 | 3.8 | 3.5 | 4.0 |
| impression | | | | | | | | |

1. Formulation containing a polyphenol-containing composition and isomaltulose.

2. Formulation according to claim 1, wherein the polyphenol-containing composition has a fraction of at least 90% by weight, preferably at least 94% by weight, of EGCG, based on the weight of the dry matter of the polyphenol-containing composition.

3. Formulation according to claim 1 or 2, wherein the polyphenol-containing composition has a fraction of at most 2.5% by weight of caffeine, preferably at most 0.1% by weight, in each case based on the weight of the dry matter of the polyphenol-containing composition.

4. Formulation according to one of the preceding claims, wherein the polyphenol-containing composition has a fraction of at most 2.5% by weight of gallocatechin gallate (GCG), based on the weight of the dry matter of the polyphenol-containing composition.

5. Formulation according to one of the preceding claims, wherein the polyphenol-containing composition has a fraction of at most 5.0% by weight of epicatechin gallate (ECG), preferably at most 3.0% by weight, in each case based on the weight of the dry matter of the polyphenol-containing composition.

12. Use of a formulation according to one or more of claims 1 to 10 for supporting fat metabolism in mammals, in particular in humans.

13. Use of a formulation according to one or more of claims 1 to 10 for reducing the fat mass in mammals, in particular in humans.

14. Use of isomaltulose in a formulation containing a polyphenol-containing composition, in particular a formulation according to one of claims 1 to 10 for taste masking.

15. Use according to claim 14, wherein the taste masking is taste masking of a polyphenol-containing composition.

16. Use according to one of claims 11 to 15, wherein the polyphenol-containing composition is a plant extract.

17. Use according to one of claims 11 to 16, wherein the plant extract is an extract of black tea, an extract of Oolong tea or an extract of green tea.

18. Use according to one of claims 11 to 17, wherein the extract contains additional natural or nature-identical odor and/or taste substances.

19. Use according to one of claims 11 to 18, wherein the polyphenol-containing composition is epigallocatechin gallate (EGCG).

20. Use according to one of claims 11 to 19, wherein the EGCG is present in the polyphenol-containing composition in a fraction of at least 90% by weight, preferably at least 94% by weight (in each case based on the dry matter of the polyphenol-containing composition).

21. Use according to one of claims 11 to 20, wherein the fraction of polyphenol-containing composition in the formulation is 0.1 to 10% by weight (based on the dry matter of the formulation).

22. Use according to one of claims 11 to 21, wherein the fraction of polyphenol-containing composition in the formulation is 0.3 to 5% by weight (based on the dry matter of the formulation).

23. Use according to one of claims 11 to 22, wherein the fraction of isomaltulose in the formulation is 30 to 85% by weight (based on the dry matter of the formulation).

24. Use according to one of claims 11 to 23, wherein the fraction of isomaltulose in the formulation is 40 to 70% by weight (based on the dry matter of the formulation).

25. Use according to one of claims 11 to 24, wherein the fraction of isomaltulose in the formulation is 50 to 61% by weight (based on the dry matter of the formulation).

26. Use according to one of claims 11 to 25, wherein the formulation is an instant powder, a dry flavor formula, a drink powder, a food, an article consumed for pleasure or a medicament.

27. Use according to one of claims 11 to 26, wherein the formulation contains food-compatible acids, food-compatible salts, intense sweeteners, taste substances, flavor or odor substances, a further sweetening agent and/or caffeine.

28. Use according to one of claims 11 to 27, wherein the fraction of food-compatible acids in the formulation is 5 to 10% by weight (based on the dry matter of the formulation).

29. Use according to one of claims 11 to 28, wherein the fraction of intense sweeteners in the formulation is 0.1 to 1% by weight (based on the dry matter of the formulation).

30. Use according to one of claims 11 to 29, wherein the fraction of taste substances in the formulation is 0.5 to 30% by weight (based on the dry matter of the formulation).

31. Use according to one of claims 11 to 30, wherein the formulation is in dry form.

32. Use according to one of claims 11 to 31, wherein the formulation is in freeze-dried, agglomerated or pulverized form.

33. Use according to one of claims 11 to 32, wherein the formulation contains flow enhancers, in particular silica.

34. Use according to one of claims 11 to 33, wherein the formulation contains natural and/or synthetic dyes.

35. Use according to one of claims 11 to 34, wherein the formulation contains natural or synthetic vitamins.

36. Use according to one of claims 11 to 35, wherein the formulation contains minerals and trace elements.

37. Instant powder containing 50 to 80% by weight isomaltulose, 0.1 to 10% by weight EGCG, 0.1 to 10% by weight caffeine, and also 0.005 to 1.0% by weight B vitamins.
